(19) United States
(12) Patent Application Publication
(10) Pub. No.: US 2022/0347434 A1
(43) Pub. Date: Nov. 3, 2022

(54) DEFLECTABLE SHAFTS FOR DELIVERY SYSTEMS

(71) Applicant: Edwards Lifesciences Corporation, Irvine, CA (US)

(72) Inventors: Nasser William Saleh, Stockton, CA (US); Salvatore Manzella, JR., Aliso Viejo, CA (US); Salomon Xavier Valencia, Aliso Viejo, CA (US)

(21) Appl. No.: 17/811,737

(22) Filed: Jul. 11, 2022

Related U.S. Application Data
(63) Continuation of application No. PCT/US2021/013082, filed on Jan. 12, 2021.
(60) Provisional application No. 62/962,026, filed on Jan. 16, 2020.

(51) Int. Cl. A61M 25/01

(52) U.S. Cl. CPC A61M 25/0147; A61M 25/0138

(57) ABSTRACT

The present disclosure generally relates to systems, devices, and methods for delivery systems including deflectable elongate shafts. A delivery system may include a deflectable elongate shaft including a pull tether extending along the length of the elongate shaft and having a distal portion and a proximal portion and an intermediate portion, with the distal portion of the pull tether coupled to a portion of the elongate shaft such that the intermediate portion of the pull tether may variably move towards the first set of bi-directional cuts or the second set of bi-directional cuts. The pull tether may be configured to be pulled to deflect the hypotube.

### CROSS-REFERENCE TO RELATED APPLICATIONS

This application is a continuation of PCT patent application no. PCT/US2021/013082 filed on Jan. 12, 2021, which claims priority to and the benefit of U.S. Provisional Application No. 62/962,026 filed on Jan. 16, 2020, each of these applications being incorporated herein by reference in its entirety.

### TECHNICAL FIELD

The present disclosure describes systems, devices, and methods related to implant deployment in fluidic systems and deflecting mechanisms.

### BACKGROUND

A variety of maladies may affect an individual's body. Such maladies may be of the individual's heart, and may include maladies of the individual's heart valves, including the aortic, mitral, tricuspid, and pulmonary valves. Stenosis, for example, is a common and serious valve disease that may affect the operation of the heart valves and an individual's overall well-being.

Implants may be provided that may replace or repair portions of a heart. Prosthetic implants, such as prosthetic heart valves, may be provided to replace a portion of a heart. Prosthetic aortic, mitral, tricuspid, and even pulmonary valves may be provided.

Implants may be deployed to the desired portion of the subject percutaneously, in a minimally invasive manner. Such deployment may occur transcatheter, in which a catheter may be deployed through the vasculature of an individual.

The path to the delivery site in the subject may be tortuous. As such, an elongate shaft for a delivery apparatus is preferably deflectable, to allow for the elongate shaft to deflect to accommodate the path within the subject. A variety of issues may arise as the elongate shaft deflects though, including incorrect orientation within the subject as well as possible damage to the elongate shaft. Further control of the deflection of the elongate shaft may be desirable as well.

### SUMMARY

The present systems and methods relate to delivery systems and may relate to deflectable elongate shafts for such delivery systems within a subject in various procedures, including (but not limited to) medical and training procedures. Such filtering may occur as part of a deployment system for an implant within a subject. Subjects include (but are not limited to) medical patients, veterinary patients, animal models, cadavers, and simulators of the cardiac and vasculature system (e.g., anthropomorphic phantoms and explant tissue).

In embodiments herein, a delivery system for an implant may be provided. The delivery system may include an elongate shaft having a length and having an implant retention area for retaining an implant. A hypotube may extend along the length of the elongate shaft and may have a proximal portion and a distal portion, and may include a first set of bi-directional cuts aligned longitudinally along a first side of the hypotube and a second set of bi-directional cuts aligned longitudinally along a second side of the hypotube that is opposite the first side, and two longitudinally extending spines each positioned on opposite sides of the hypotube between the first set of bi-directional cuts and the second set of bi-directional cuts.

A pull tether may extend along the length of the elongate shaft and may have a distal portion and a proximal portion and an intermediate portion, with the distal portion of the pull tether coupled to a portion of the elongate shaft such that the intermediate portion of the pull tether may variably move towards the first set of bi-directional cuts or the second set of bi-directional cuts, the pull tether configured to be pulled to deflect the hypotube.

In embodiments herein, a delivery system for an implant may be provided. The delivery system may include an elongate shaft having a length. The elongate shaft may have an implant retention area for retaining the implant, a first hypotube extending along the length of the elongate shaft and including a proximal end and a distal end, and a plurality of cuts configured to allow the first hypotube to deflect in a first direction.

The elongate shaft may include a second hypotube including a distal portion and a proximal portion, the distal portion extending over the first hypotube and including a plurality of cuts configured to allow the second hypotube to deflect in a second direction that is opposite the first direction, and the proximal portion being positioned proximal of the distal end of the first hypotube and including a plurality of cuts configured to allow the proximal portion to deflect in both the first direction and the second direction.

In embodiments herein, a method may be provided. The method may include inserting an elongate shaft of a delivery apparatus into vasculature of a subject. The elongate shaft may have a length and may include an implant retention area for retaining an implant, a hypotube extending along the length of the elongate shaft and including a first set of bi-directional cuts aligned longitudinally along a first side of the hypotube and a second set of bi-directional cuts aligned longitudinally along a second side of the hypotube that is opposite the first side, and two longitudinally extending spines each positioned on opposite sides of the hypotube between the first set of bi-directional cuts and the second set of bi-directional cuts. A pull tether may extend within at least a portion of the hypotube.

The method may include causing the pull tether to deflect within the hypo tube towards either the first set of bi-directional cuts or towards the second set of bi-directional cuts.

The method may include passively deflecting the hypotube to cause the pull tether to deflect within the hypotube towards either the first set of bi-directional cuts or towards the second set of bi-directional cuts.

The pull tether may be configured to deflect towards a first direction to deflect towards the first set of bi-directional cuts and may be configured to deflect towards a second direction to deflect towards the second set of bi-directional cuts, and further comprising pulling the pull tether proximally to deflect the hypotube towards either the first direction or the second direction.

The method may include pulling the pull tether to deflect the hypotube towards the first direction if the pull tether is caused to deflect towards the first direction or towards the second direction if the pull tether is caused to deflect towards the second direction.

The pull tether may be aligned with at least one of the two longitudinally extending spines prior to being deflected within the hypotube towards either the first set of bi-directional cuts or towards the second set of bi-directional cuts.

In embodiments herein, a method may be provided. The method may include inserting an elongate shaft of a delivery apparatus into vasculature of a subject. The elongate shaft may have a length and may include an implant retention area for retaining an implant. A first hypotube may extend along the length of the elongate shaft and include a proximal end and a distal end, and a plurality of cuts configured to allow the first hypotube to deflect in a first direction.

A second hypotube may include a distal portion and a proximal portion, the distal portion extending over the first hypotube and including a plurality of cuts configured to allow the second hypotube to deflect in a second direction that is opposite the first direction, and the proximal portion being positioned proximal of the distal end of the first hypotube and including a plurality of cuts configured to allow the proximal portion to deflect in both the first direction and the second direction.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar embodiments.

FIG. 1 is a side view of a delivery apparatus.

FIG. 2 is a perspective view of the handle of the delivery apparatus shown in FIG. 1.

FIG. 3 is a side exploded assembly view of components of the delivery apparatus shown in FIG. 1.

FIG. 4 is a side exploded assembly view of components of the delivery apparatus shown in FIG. 1.

FIG. 5 is a bottom view of a hypotube according to an embodiment of the present disclosure.

FIG. 6 is a bottom view of a hypotube according to an embodiment of the present disclosure.

FIG. 7 is a top view of the hypotube shown in FIG. 6.

FIG. 8 is a side cross sectional view of the hypotube shown in FIGS. 6 and 7 positioned within the hypotube shown in FIG. 5.

FIG. 9 is a side cross sectional view of the hypotube shown in FIGS. 6 and 7 positioned within the hypotube shown in FIG. 5.

FIG. 10 is a perspective view of a prosthetic valve.

FIG. 11 is a side schematic view of a delivery apparatus approaching an aortic valve.

FIG. 12 is a side schematic view of a prosthetic aortic valve in position.

FIG. 13 is a top view of a hypotube according to an embodiment of the present disclosure.

FIG. 14 is a side view of the hypotube shown in FIG. 13.

FIG. 15 is a side view of a hypotube according to an embodiment of the present disclosure.

FIG. 16 is a top view of the hypotube shown in FIG. 15.

FIG. 17 is an end view of the hypotube shown in FIG. 15.

FIG. 18 is a side view of a hypotube according to an embodiment of the present disclosure.

FIG. 19 is a side view of a hypotube according to an embodiment of the present disclosure.

FIG. 20 is a top view of the hypotube shown in FIG. 19.

FIG. 21 is a side view of a hypotube according to an embodiment of the present disclosure.

### DETAILED DESCRIPTION

The following description and examples illustrate some example embodiments of the disclosure in detail. Those of skill in the art will recognize that there are numerous variations and modifications of the disclosure that are encompassed by its scope. Accordingly, the description of a certain example embodiment should not be deemed to limit the scope of the present disclosure.

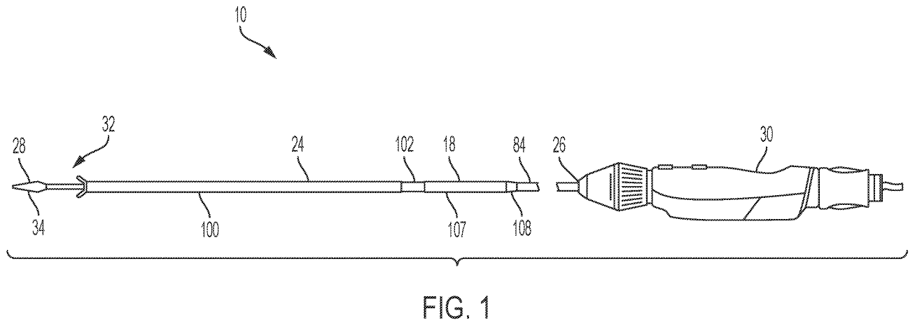

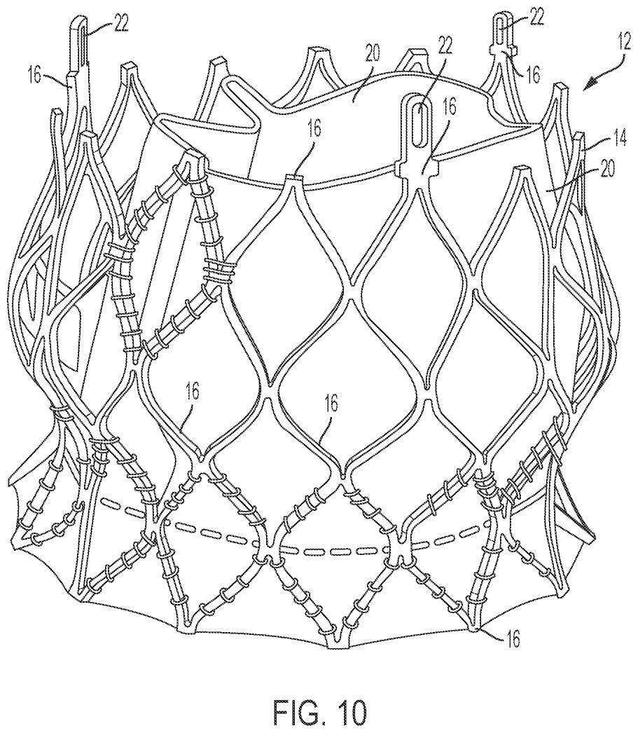

FIG. 1 illustrates an embodiment of a delivery system 10 for an implant, which may be similar to the implant 12 marked in FIG. 10. The implant 12 may be an aortic implant comprising a prosthetic aortic valve. In embodiments, the implant may have other forms than shown in FIG. 10, for example the implant may be a mitral, tricuspid, or pulmonary prosthetic valve, among other forms of prosthetics. The implant may comprise a stent, clip, or other form of implant that may be inserted in a portion of the subject, including the heart.

The implant 12 may be an expandable implant as shown in FIG. 10, which may be configured to be expanded to be placed in position within the native valve location. The implant 12 may include a frame 14 including a plurality of supports 16 configured to be compressed for positioning within the delivery apparatus 18 and configured to be expanded at the desired time. The frame 14 may support prosthetic valve leaflets 20 that operate in lieu of the native valve leaflets. The frame 14 may include couplers 22 for coupling to the delivery apparatus 18, to retain the implant 12 to the delivery apparatus 18 until deployment is desired. The couplers 22 may comprise apertures as shown in FIG. 10, or may have other forms as desired. Although implant 12 is shown in FIG. 10, the use of the delivery apparatus 18 is not limited to the embodiment of implant 12 shown in FIG. 10, and may extend to other forms of implants as desired.

Referring back to FIG. 1, the delivery system 10 may include a delivery apparatus 18 that may include an elongate shaft 24 including a proximal end 26 and a distal end 28 and having a length between the proximal end 26 and the distal end 28. A housing in the form of a handle 30 may be positioned at a proximal portion of the elongate shaft 24 including the proximal end 26 of the elongate shaft 24. The handle 30 may be configured for an individual to grip to utilize when operating the delivery apparatus 18. The elongate shaft 24 may extend outward from the handle 30 and may be configured to be inserted into a subject to be directed to a desired treatment site of the subject. The elongate shaft 24 may be configured to be inserted into the vasculature of the subject, or otherwise may be inserted into the vasculature of the subject. Such insertion may be percutaneous and minimally invasive, such as transfemoral entry. Other forms of entry, such as transapical may be utilized as well. The handle 30 remains exterior to the subject during insertion.

The elongate shaft 24 may include an implant retention area 32, which in the embodiment disclosed herein may be covered by a sheath to form a capsule. The implant retention area 32 may be configured to retain the implant. The implant may be retained in the implant retention area 32 until the desired time for deployment of the implant. The elongate shaft 24 may be inserted into the subject and navigated to the desired deployment location to position the implant retention area 32 as desired. The delivery apparatus 18 may then be operated to deploy the implant from the implant retention area 32.

The elongate shaft 24 may further include a nose cone 34 at the distal end 28 of the elongate shaft 24. The nose cone 34 may form the tip of the elongate shaft 24 and may be pliable to avoid injury to portions of the subject contacted by the tip of the elongate shaft 24.

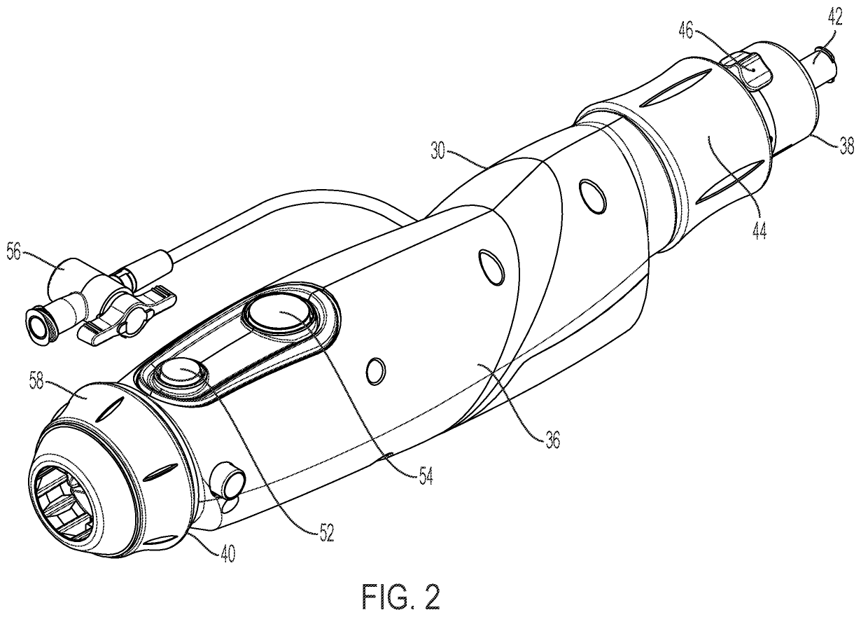

FIG. 2 illustrates a perspective view of the handle 30 of the delivery apparatus 18. The handle 30 may include an outer surface 36 for being gripped, and may include a proximal portion 38 and a distal portion 40. The outer surface 36 of the handle 30 may be configured to be ergonomic, to be gripped by the user. The handle 30 may be configured to be driven distally to move the elongate shaft 24 distally, or may be driven proximally to move the elongate shaft 24 proximally. The handle 30 may be configured to be rotated about the longitudinal axis of the handle 30 and the elongate shaft 24 to rotate and torque the elongate shaft 24. Such rotation may be desired to provide for a desired orientation of the implant to be deployed from the implant retention area 32. The proximal portion 38 may include a flush port 42 for flushing fluid (including air) from the elongate shaft 24.

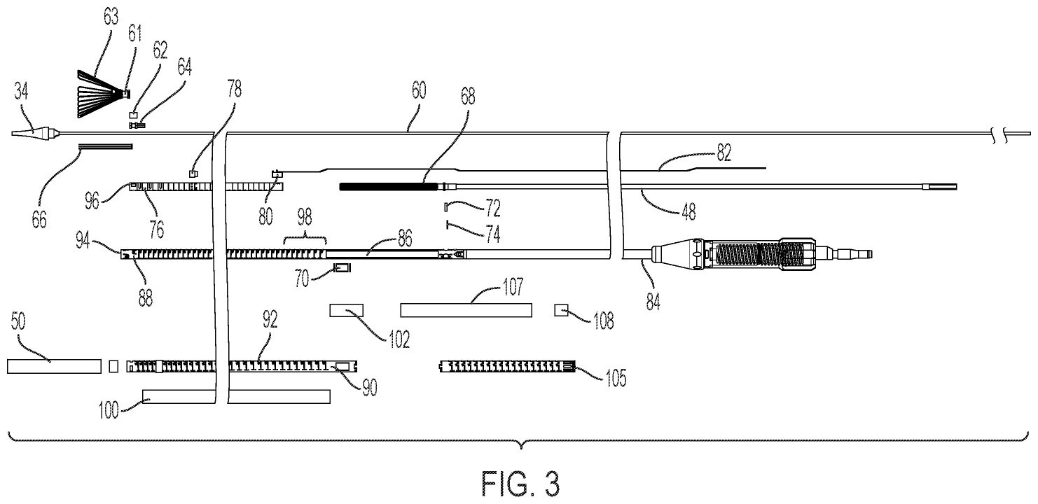

The handle 30 may further include a release mechanism positioned therein, which may be configured to release the implant from the implant retention area 32. Components of the release mechanism may include a release actuator 44 for being operated to release the implant from the implant retention area 32. The release actuator 44 may be positioned on the proximal portion 38 of the handle 30. The release mechanism may further include a lock 46 on the proximal portion 38 for locking the release actuator 44 in position. The release mechanism may further include a motor or other driving device (such as a manually driven device) that may be positioned in the handle 30. The motor or other driving device may be configured to rotate a torque shaft 48 as shown in FIG. 3, for rotating to move an outer sheath 50 that may cover the implant retention area 32.

The release mechanism may include a control device for operating the motor or other driving device. As shown in FIG. 2, the control device may be in the form of buttons 52, 54 or other form of control device. One button 52 may be configured to retract the outer sheath 50 (move the outer sheath 50 proximally) and another button 54 may be configured to advance the outer sheath 50 (move the outer sheath distally). As such, the control device may be used to selectively control deployment of the implant from the implant retention area 32 by movement of the outer sheath 50 (e.g., either exposing the implant for release or covering the implant for recapture).

The handle 30 may further include a flush valve 56 for allowing fluids to be flushed from the elongate shaft 24.

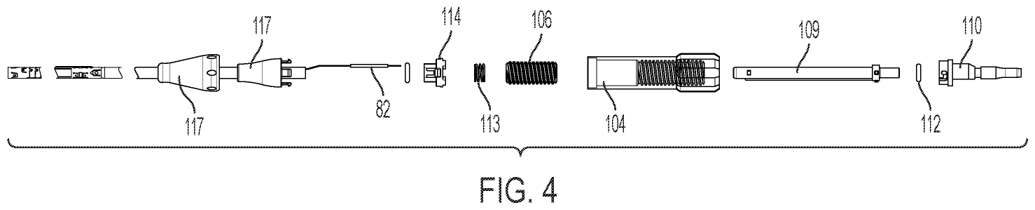

The handle 30 may include a deflection mechanism 58 configured to cause at least a portion of the elongate shaft 24 to deflect. The deflection may be in longitudinal planes extending outward from the longitudinal axis of the elongate shaft 24. Such deflection may be utilized to accommodate various bends in the subject's anatomy that may need to be navigated to deliver the implant to the desired location. The deflection mechanism 58 may provide for a controllable deflection of the elongate shaft 24, as opposed to a passive deflection that may occur by simply passing a flexible shaft through bends in the subject's anatomy. The deflection mechanism 58 is discussed in greater detail in regard to FIG. 4.

FIG. 3 illustrates an exploded assembly view of components of the delivery apparatus 18, including the elongate shaft 24. The uppermost components shown in FIG. 3 are generally the innermost components of the elongate shaft 24 and the lowermost components shown in FIG. 3 are generally the outermost components of the elongate shaft 24.

Referring to FIG. 3, the elongate shaft 24 may comprise an assembly of components. Various components may comprise sub-assemblies of the elongate shaft 24. The elongate shaft 24 may include multiple layers or multiple sheaths extending over other layers or sheaths.

In the embodiment shown in FIG. 3, an innermost component of the elongate shaft 24 may comprise an inner shaft or guidewire lumen 60. The guidewire lumen 60 may extend for the length of the elongate shaft 24 and may have a proximal end that couples to the handle 30. A distal end of the guidewire lumen 60 may couple to the nose cone 34. A manifold 61 may be coupled to the guidewire lumen 60 and retain sutures 63 for retaining the implant in position within the implant retention area 32. A retainer 62, 64 may be coupled to the guidewire lumen 60 for coupling to release pins 66. Upon movement of the release pins 66, the implant may be allowed to deploy from the implant retention area 32.

A torque shaft 48 may be provided comprising a sheath that extends over the guidewire lumen 60. The torque shaft 48 may have a proximal end that couples to the motor or other driving device of the handle 30, and may have a distal portion 68 that comprises a threaded portion for a threaded body 70 (such as a nut or other form of threaded body) to slide along. Washers 72, 74 may hold the torque shaft 48 in position.

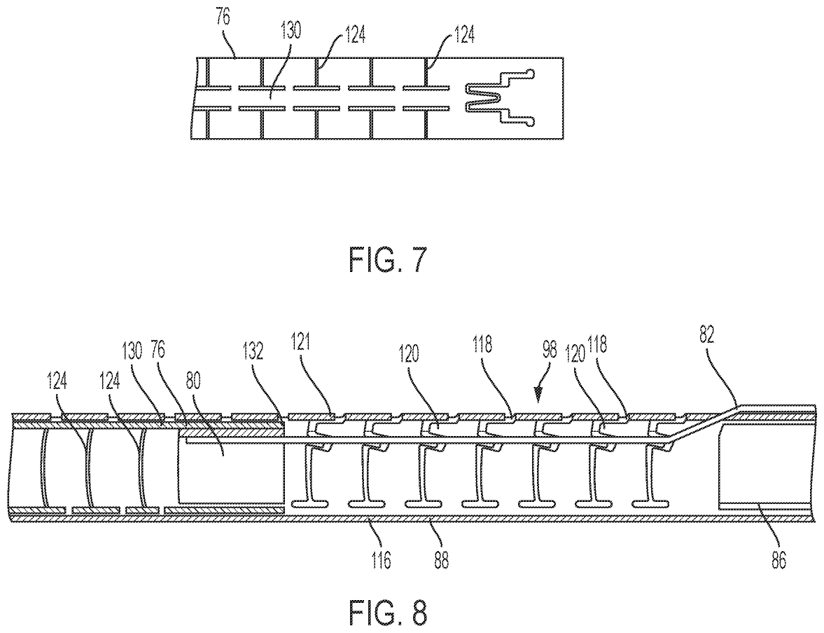

A hypotube 76 may be positioned distal of the distal end of the torque shaft 48 and may be spaced from the distal end of the torque shaft 48 as shown in FIG. 3. The hypotube 76 may be configured to extend along the length of the elongate shaft 24 and may include a proximal end and a distal end. The hypotube 76 may include a plurality of cuts as shown in FIGS. 6 and 7 that may allow the hypotube 76 to deflect in a direction. A stop 78 for the retainer 62, 64 may be positioned within an interior cavity of the hypotube 76, to prevent undesired proximal movement of the retainer 62, 64 within the hypotube 76. The hypotube 76 may comprise a sheath extending over the guidewire lumen 60.

A pull tether coupler 80 may be positioned within the hypotube 76. The pull tether coupler 80 may comprise a body configured to couple to a distal end of a pull tether 82 and retain the pull tether 82 to the hypotube 76. The pull tether 82 may have a distal end coupled to the pull tether coupler 80 and a proximal portion including a proximal end coupled to the deflection mechanism.

A flex shaft 84 may comprise a sheath extending over the torque shaft 48 and may have a proximal end coupled to the deflection mechanism and may have a distal end coupled to a proximal end of a torque shaft channel 86. The flex shaft 84 may comprise the substantial length of the elongate shaft 24 and forms an outer surface of the elongate shaft 24 as marked in FIG. 1. The flex shaft 84 may be configured to be flexible to accommodate the anatomy of the subject being entered.

A hypotube 88 may have a proximal portion including the torque shaft channel 86. The torque shaft channel 86 may comprise an opening allowing the threaded body 70 to transmit motion provided from the threaded portion of the torque shaft 48 to the outer sheath 50. The threaded body 70 may slide longitudinally along the threaded portion of the torque shaft 48 because the torque shaft channel 86 may prevent rotation of the threaded body 70. The threaded body 70 may extend outward through the torque shaft channel 86 to couple to a proximal portion 90 of hypotube 92, and thus cause hypotube 92 to slide longitudinally along with the threaded body 70. As such, the outer sheath 50 is coupled to the hypotube 92 and is slid as well.

The hypotube 88 may have a distal portion including a distal end 94 that couples to a distal end 96 of the hypotube 76. As such, the hypotube 88 may couple to the pull tether 82 and the hypotube 76 at the distal end 94 of the hypotube 88. A proximal portion 98 of the hypotube adjacent the torque shaft channel 86 does not cover the hypotube 76. The proximal portion 98 of the hypotube may include the proximal end of the hypotube.

The outer sheath of the elongate shaft 24 may include multiple components. Such components may include the outer sheath 50 extending over the implant retention area 32. A proximal end of the outer sheath 50 may couple to the hypotube 92, which may include a sheath 100 extending over the hypotube 92 and forming an outer surface of the elongate shaft 24 as marked in FIG. 1. The proximal end of the hypotube 92 may couple to the threaded body 70, and such connection may be covered with an outer sheath 102 forming an outer surface of the elongate shaft 24 as marked in FIG. 1. Further, a proximal portion of the outer sheath may include a hypotube 105, which may impede fluid (such as blood) from entering into the torque shaft channel 86 when the outer sheath is slid distally. The hypotube 105 may be covered with outer sheaths 107, 108 that form an outer surface of the elongate shaft 24 as marked in FIG. 1.

In operation, the release mechanism may operate to rotate the torque shaft 48, which causes the threaded body 70 to slide longitudinally along the torque shaft channel 86. This longitudinal motion is provided to the outer sheath 50, which causes the implant to be exposed and deployed from the elongate shaft 24.

The flexibility and deflection of the elongate shaft 24 will be discussed in regard to the deflection mechanism shown in FIG. 4 and other features of the system disclosed herein. Referring to FIG. 4, a deflection mechanism may be provided that operates to actively deflect at least a portion of the elongate shaft 24. The deflection mechanism may be positioned at a proximal end of the elongate shaft 24. The deflection mechanism may be positioned distal the handle 30 and at the proximate end of the elongate shaft 24, although other positions may be utilized as desired.

The deflection mechanism may include a control device 104 that may be operated by an individual to control the deflection of the elongate shaft. The control device 104 is shown to comprise a rotatable knob in FIG. 4, although other forms of control devices may be utilized as desired. The control device 104 may include threading on an interior of the control device 104 that engages a threaded body 106. The threaded body 106 may configured to slide along a rail 109 that impedes rotational movement of the threaded body 106, thus causing the threaded body 106 to slide along the length of the threaded body 106. A proximal mounting 110 and sealing ring 112 may be configured to couple a proximal end of the deflection mechanism to the handle 30.

A distal portion of the threaded body 106 may couple to a spring 113 and coupler 114, with the coupler 114 coupling the threaded body 106 to the proximal end of the pull tether 82. Thus, longitudinal movement of the threaded body 106 longitudinally moves the pull tether 82. A distal mounting 117 couples the deflection mechanism to the proximal end of the flex shaft 84.

In operation, the control device 104 may be operated to move the pull tether 82 proximally or distally, to vary the operational length of the pull tether 82. Proximal movement of the pull tether 82 may shorten the operational length of the pull tether 82, thus causing the elongate shaft 24 to flex at the portions of the elongate shaft 24 proximal the distal coupling point of the pull tether 82 on the elongate shaft 24.

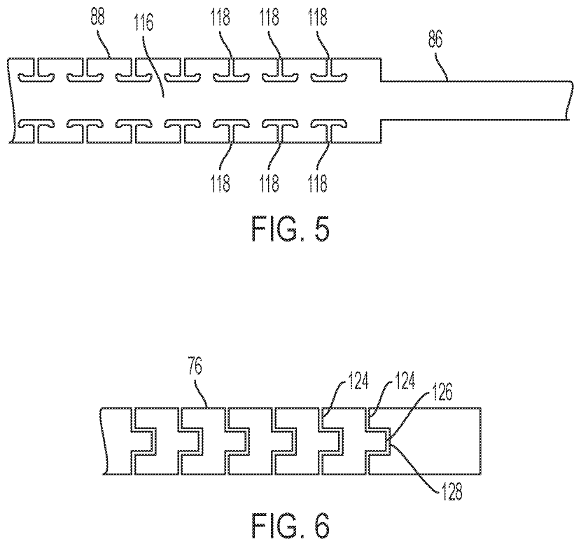

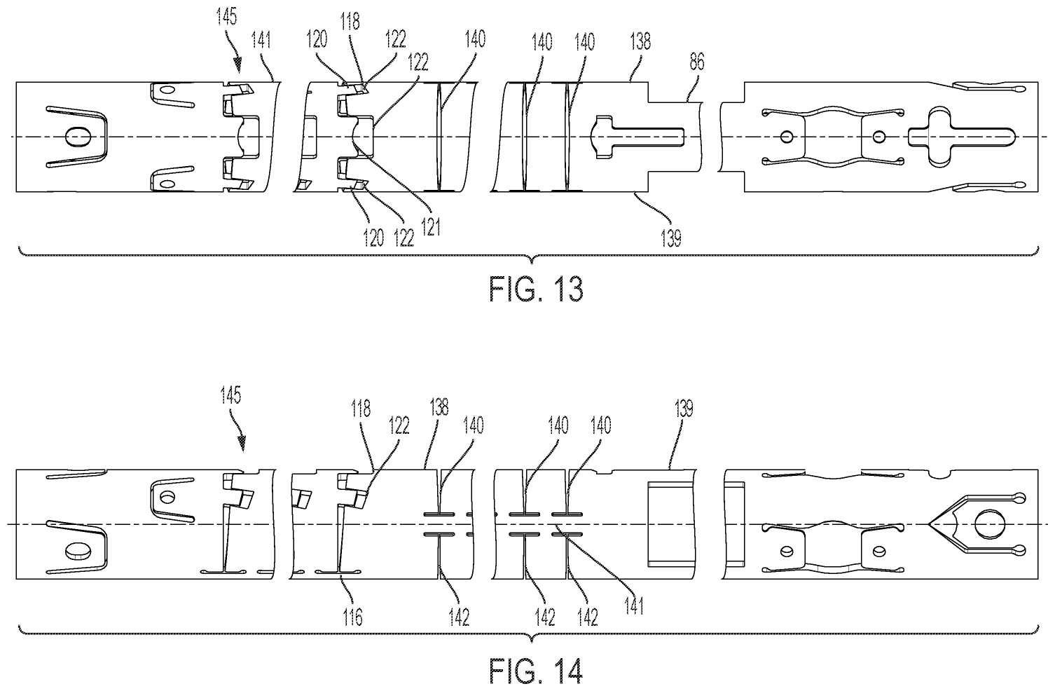

FIG. 5 illustrates a bottom view of a proximal portion of the hypotube 88. The hypotube 88 may include a single spine 116 extending longitudinally along the length of the hypotube 88. The single spine 116 may be positioned on a side of the hypotube 88 that the hypotube 88 is not intended to deflect towards. Cuts 118 may extend through the surface of the hypotube 88, extending from the outer surface to the inner surface facing the central cavity of the hypotube 88. The cuts may extend around the outer circumference of the hypotube 88 aside from the location of the single spine 116. FIG. 13 illustrates with reference number 118 the cut pattern of the opposite side of the hypotube 88. The cut 118 may include a plurality of teeth 120 configured to fit into and engage recesses 122 having corresponding shapes as the teeth 120. A central tooth 121 may be larger than the two side teeth 120. As such, the cuts 118 may provide for a single direction of deflection for the hypotube 88, namely in a direction towards the central tooth 121 represented in FIG. 13. FIG. 14 illustrates a side view of a cut 118 that the hypotube 88 may utilize along its length.

FIG. 6 illustrates a bottom view of a cut pattern of the inner hypotube 76. The inner hypotube 76 may include cuts 124 forming a tooth 126 that engages a correspondingly shaped recess 128. The cuts 124 may be configured for the inner hypotube 76 to deflect in a single direction. The size of the gap between the tooth 126 and the recess 128 may be smaller than for the hypotube 88, and thus less flex may be available for the inner hypotube 76 than for the hypotube 88. FIG. 7 illustrates an opposite side of the hypotube 76 showing that a single spine 130 extends longitudinally along the length of the hypotube 76.

The outer hypotube 88 may extend over the inner hypotube 76 as discussed in regard to FIG. 3. FIG. 8 illustrates a close-up cross sectional view of the proximal portion 98 of the outer hypotube 88 adjacent the torque shaft channel 86 as discussed in regard to FIG. 3. Certain components of the elongate shaft 24 are excluded from view for clarity. The hypotube 88 extends over the inner hypotube 76 and particularly over the proximal end 132 of the hypotube 76. Thus, a portion 98 of the outer hypotube 88 is positioned between the proximal end 132 of the inner hypotube 76 and the torque shaft channel 86 that does not cover the inner hypotube 76. The portion 98 is positioned distal a portion of the elongate shaft comprising the channel 86 and the torque shaft 48, which may have a greater stiffness than the proximal portion of the outer hypotube 88.

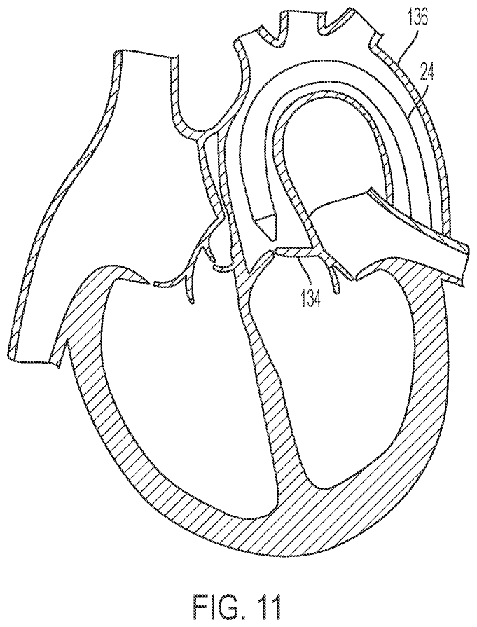

A distal portion of the hypotube 88 extends over the hypotube 76 and includes a plurality of cuts configured to allow the hypotube 88 to deflect in a direction that is opposite the direction of deflection of the hypotube 76. The spines 116, 130 of the respective hypotubes 88, 76 are positioned 180 degrees from each other. As such, the inner hypotube 76 may impede deflection of the outer hypotube 88 in a direction away from the central tooth 121 of the outer hypotube 88. Such a feature may serve to prevent kinks from forming in the outer hypotube 88 if the outer hypotube 88 were possibly deflected in the wrong direction, as discussed further in regard to FIG. 11.

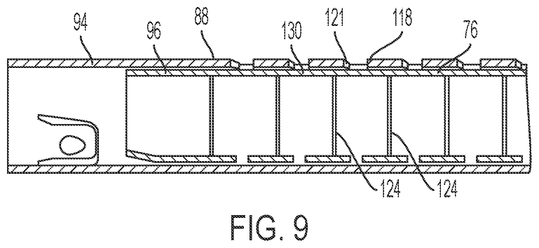

FIG. 9 illustrates a close-up cross sectional view of the distal end 96 of the inner hypotube 76 coupled to the distal end 94 of the outer hypotube 88. A coupler or the like may couple the distal end 96 of the inner hypotube 76 to the distal end 94 of the outer hypotube 88. The coupling between the distal ends 96, 94 transmits the force from the pull tether 82 to the hypotube 88.

Features of the deflection of the elongate shaft 24 will be discussed in regard to FIG. 11. FIG. 11 illustrates a view of the elongate shaft 24 as it is being delivered to the aortic valve 134 of the heart. Notably, the elongate shaft 24 has deflected to account for the geometry of the heart, and particularly the aortic arch 136. The deflection mechanism has operated to pull the pull tether 82 proximally, causing the elongate shaft 24 to deflect.

The construction of the elongate shaft 24, however, may provide difficulties if the direction of deflection of the outer hypotube 88 is not inserted into the aortic arch, or another portion of the subject's anatomy, in the proper orientation. For instance, upon insertion of the elongate shaft 24 into the subject's anatomy, the elongate shaft may have rotated about its longitudinal axis and thus may have the direction of deflection of the outer hypotube 88 in an opposite direction than a desired deflection, or in a generally different direction of deflection than is desired.

As such, referring to FIG. 11, if the elongate shaft 24 is advanced through the aortic arch or other portion of the subject with the direction of deflection of the outer hypotube 88 in the wrong direction, then damage to the elongate shaft 24 may occur. For example, the elongate shaft 24 may be inserted into the aortic arch 136 and may contact a wall of the aortic arch 136. Such contact may deflect the elongate shaft 24 in a direction opposite to the direction of deflection of the outer hypotube 88. The presence of the inner hypotube 76 along the distal portion of the outer hypotube 88 may prevent kinking at those portions, but the portion 98 of the outer hypotube 88 shown in FIG. 8 may kink due to the lack of presence of the inner hypotube 76 at that portion 98.

FIGS. 13 and 14 illustrate a variation on the outer hypotube 88, in which hypotube 138 shown includes the cut pattern of the hypotube 88 along a distal portion 145 of the hypotube 138, but varies the cut pattern at a proximal portion 139 of the hypotube 138 where the inner hypotube 76 is not present (corresponding to portion 98 discussed). The hypotube 138 may include a distal portion 145 and a proximal portion 139, with the distal portion 145 configured to extend over the hypotube 76 and including the cut pattern of the hypotube 88, which allows the hypotube 138 to deflect in a direction (e.g., a second direction) that is opposite the direction (e.g., a first direction) of deflection of the inner hypotube 76. The distal portion 145 may include a single spine 116 that is positioned between the plurality of cuts 118 of the hypotube 138. The spine 116 may be at a position that is 180 degrees from the spine 130 of the inner hypotube 76.

The proximal portion 139 may be positioned proximal of the distal end of the inner hypotube 76 and may include a plurality of cuts 140, 142 that are configured to allow the proximal portion 139 to deflect in both the second direction of the distal portion 145 and the first direction of the inner hypotube 76. FIGS. 13 and 14 illustrate that the cuts of the proximal portion 139 of the outer hypotube 138 may be provided as first and second sets of bi-directional cuts (with a first set of cuts 140 and a second set of cuts 142). The bi-directional cuts 140, 142 are bi-directional because they allow for flex in two directions in the same plane. The cuts 140, 142 may be longitudinally aligned as shown in FIG. 13, and as represented in FIG. 14, two spines 141 (a mirror image of FIG. 14 is provided on the other side of FIG. 14) separate the sets of longitudinally aligned cuts 140, 142. A first set of the cuts 140 may be positioned on one side of the hypotube 138, and a second set of the cuts 142 may be positioned on an opposite side of the hypotube 138. The first set of cuts 140 may be aligned longitudinally along a first side of the hypotube 138 and the second set of cuts 142 may be aligned longitudinally along a second side of the hypotube 138 that is opposite the first side. The spines 141 may be positioned rotationally orthogonal to the cuts 140, 142 and may extend parallel with each other. The spines 141 may be longitudinally extending and positioned on opposite sides of the hypotube 138 between the first set of cuts 140 and the second set of cuts 142. The spines 141 may be rotationally offset from the spine 116 of the cuts 118 of the distal portion of the hypotube 138 by 90 degrees. As such, the dual spine 141 configuration and uniformly shaped cuts 140 on opposite sides of the hypotube 138 may allow for bi-directional flex of the proximal portion of the hypotube 138.

Further, the cuts 140, 142 may each have cuts curved such that an entire interior area of the cuts 140, 142 contacts simultaneously. For example, the cuts may have a rounded oval shape as desired.

As such, referring back to FIG. 11, when the elongate shaft 24 is inserted into the vasculature of a subject, if the elongate shaft 24 is oriented in the wrong direction, then the cuts 140, 142 will allow the proximal portion 139 of the hypotube 138 to deflect in the opposite direction than the distal portion 145, thus reducing the possibility of damage to the elongate shaft 24 (including kinking). The cuts of the hypotube 138 allow the proximal portion of the hypotube 138 to deflect in the direction of deflection of the inner hypotube 76 and also in an opposite direction.

The inner hypotube 76 may yet be configured to impede the distal portion 145 of the outer hypotube 138 from deflecting in the direction of flex of the inner hypotube 76. The inner hypotube 76 may remain coupled to a pull tether 82 that is configured to be pulled proximally to deflect the outer hypotube 138.

The plurality of cuts 140, 142 of the proximal portion 139 of the outer hypotube 138 may be positioned distal the torque shaft 48 configured to move the sheath 50 for covering the implant retention area 32.

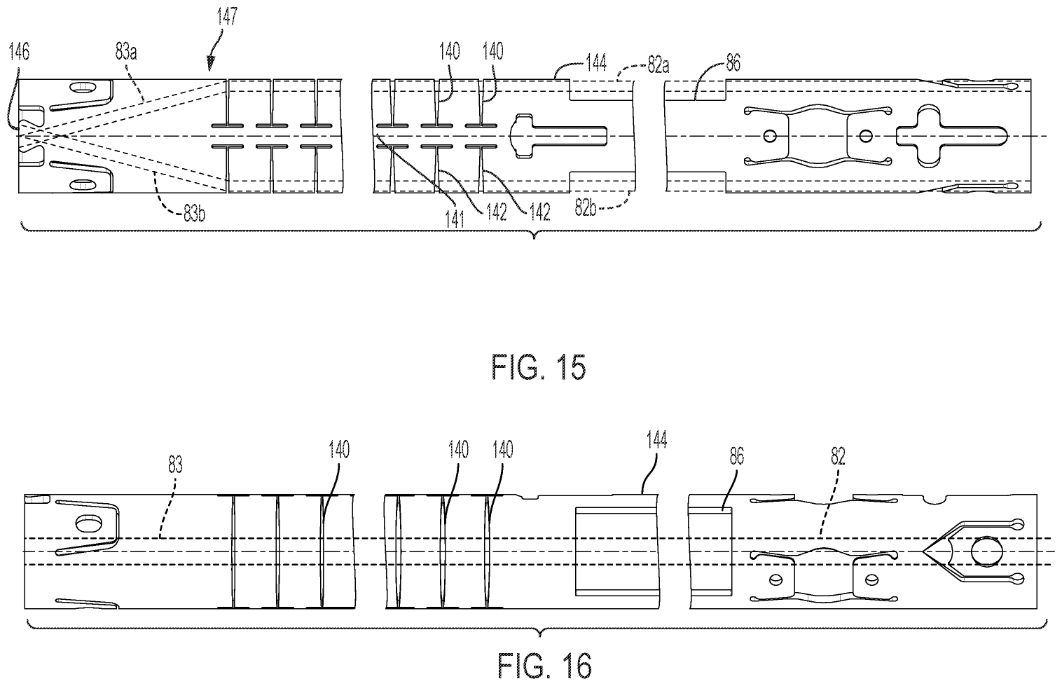

FIGS. 15 and 16 illustrate a variation of an embodiment of the outer hypotube 88 in which the pattern of cuts 140, 142 of the proximal portion 139 of the hypotube 138 shown in FIGS. 13 and 14 extend along the entire length of the hypotube 144. The hypotube 144 may be configured to deflect in two directions in a single plane. The hypotube 144 may extend along the length of the elongate shaft 24 and may have a proximal portion and a distal portion.

In such a configuration as shown in FIG. 15, the inner hypotube 76 may be excluded from the assembly of the elongate shaft 24. As such, the pull tether 82 may couple to the distal portion of the hypotube 144 and a distal portion of the elongate shaft 24. Further, the implant retention area 32 may be positioned distal of the distal portion of the hypotube 144.

The hypotube 144 may include a first set of bi-directional cuts 140 that are aligned longitudinally along a first side of the hypotube 144, and a second set of bi-directional cuts 142 aligned longitudinally along a second side of the hypotube 144 that is opposite the first side. Two longitudinally extending spines (spine 141 is marked in FIG. 15, and an opposite spine is on the opposite side than shown in FIG. 15) may each be positioned on opposite sides of the hypotube 144 between the first set of bi-directional cuts 140 and the second set of bi-directional cuts 142.

The pull tether 82 (marked with alternate pull tether positions 82a, 82b shown in FIG. 15) may extend along the length of the elongate shaft 24 and have a distal portion and a proximal portion and an intermediate portion. A proximal portion of the pull tether 82 may be coupled to a deflection mechanism, such as the deflection mechanism 58 shown in FIG. 4, that is configured to pull the pull tether 82 to deflect the hypotube 144.

A distal portion of the pull tether 82 may be coupled to a portion of the elongate shaft 24 such that an intermediate portion of the pull tether 82 is aligned with at least one of the longitudinally extending spines 141. The distal portion 147 of the hypotube 144 as shown in FIG. 15 may comprise a coupling point, which may comprise a coupler 146 at a distal end of the hypotube 144. The distal portion of the pull tether 82 may be coupled to the coupling point (in the form of coupler 146) that may be longitudinally aligned with one of the two longitudinally extending spines 141 (with the opposite spine being on a side that is opposite the side shown in FIG. 15).

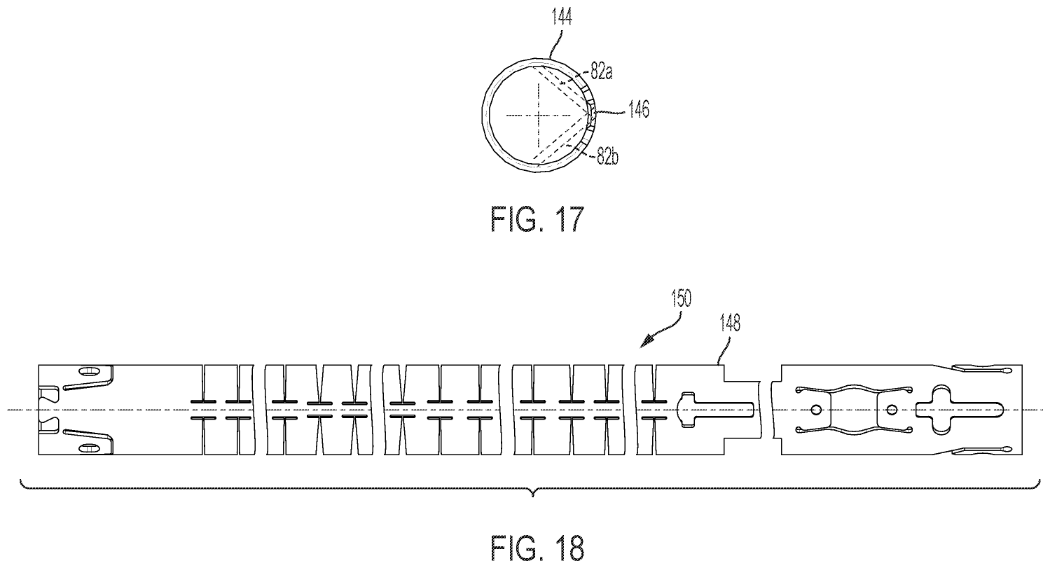

The distal portion of the pull tether 82 may be coupled to a portion of the elongate shaft 24 such that an intermediate portion of the pull tether 82 may variably move towards the first set of bi-directional cuts 140 (as marked with pull tether 82a), or the second set of bi-directional cuts 142 (as marked with pull tether 82b), and the pull tether may be configured to be pulled to deflect the hypotube 144. For example, the coupling point may be configured such that the distal portion 83 (marked with variable positions of distal portions 83a, b in FIG. 15) of the pull tether 82 may be coupled to a portion of the elongate shaft 24 such that an intermediate portion of the pull tether may variably move towards the first set of bi-directional cuts 140 or the second set of bi-directional cuts 142. Such a coupling point is shown to be intermediate the first set of cuts 140 and the second set of cuts 142, and in longitudinal alignment with the spine 141. Such a coupling point may be on a sidewall of the hypotube 88 as shown in FIGS. 15 and 17. Thus, the pull tether 82 may initially be aligned with one of the two longitudinally extending spines 141, with the distal portion of the pull tether 82 coupled to a portion of the elongate shaft 24 such that the intermediate portion of the pull tether may variably move towards the first set of bi-directional cuts 140 or the second set of bi-directional cuts 142.

In operation, as the elongate shaft 24 is inserted into and advanced through the vasculature of the subject the elongate shaft 24 may deflect due to a variety of features, such as contact with a portion of the subject or deflection over a guide wire. Due to such deflection, the hypotube 144 may deflect towards that direction. The deflection may be passive deflection, caused by contact with a surface of the subject or another feature. The pull tether 82 may thus be caused to deflect within the hypotube 144 towards the direction of deflection and be deflected towards either of the first set of cuts 140 or the second set of cuts 142 in that direction. This feature is marked in dashed lines in FIGS. 15 and 17 as the pull tether variably moving towards either the cuts 140 (as marked as pull tether 82a with distal portion 83a) or the cuts 142 (as marked as pull tether 82b with distal portion 83b). As such, the hypotube 144 may be passively deflected by the vasculature or another feature to cause the pull tether 82 to deflect within the hypotube 144 towards either the first set of cuts 140 or towards the second set of cuts 142.

Upon the pull tether moving in a direction towards those cuts 140, 142, the pull tether may be retracted proximally to continue movement in that direction. The pull tether may thus be pulled proximally to deflect the hypotube 144 towards either a first direction (which would be in a direction that is towards the cuts 140) or a second direction (which would be in a direction that is towards the cuts 142). The pull tether may deflect the hypotube 144 in a direction towards the cuts 140 if the pull tether is initially caused to deflect towards the cuts 140 and towards the first direction. Further, the pull tether may deflect the hypotube 144 in a direction towards the cuts 142 if the pull tether is initially caused to deflect towards the cuts 142 and towards the second direction. The pull tether may have be aligned with at least one of the longitudinally extending spines (such as spine 141 and the opposite spine that is on a side that is opposite the side shown in FIG. 15) prior to being deflected within the hypotube 144 towards either the first set of cuts 140 or towards the second set of cuts 142.

Thus, referring back to FIG. 11, as the elongate shaft 24 is advanced and passively deflected in a direction towards the aortic valve 134, the pull tether may move towards the cuts 140 or 142 on the inner radius of the elongate shaft 24 and thus be capable of providing active deflection in that direction. As such, the possibility of the elongate shaft 24 being rotated incorrectly is greatly reduced, as two directions of deflection in a single plane are possible in the configuration shown in FIG. 15.

FIG. 16 illustrates a top view of the configuration shown in FIG. 15. FIG. 17 illustrates an end view of the hypotube 144 shown the two possible positions of the pull tether as reference numbers 82a and 82b.

FIG. 18 illustrates a view of a hypotube 148 in which the cut pattern 150 is configured similarly as shown in FIGS. 15 and 16, yet includes cuts having different sizes. The sets of cuts may be configured to have varied cut thicknesses. The different thicknesses may allow for greater or lesser angles of deflection depending on the cut size (smaller cuts having lesser angles of deflection, and larger cuts having greater angles of deflection). As such, in embodiments, any of the cuts of the first and second sets of bi-directional cuts 140, 142 may have varied cut thicknesses as desired.

The cuts 140, 142, as disclosed, may each have cuts curved such that an entire interior area of the cuts 140, 142 contacts simultaneously. For example, the cuts may have a rounded oval shape as desired.

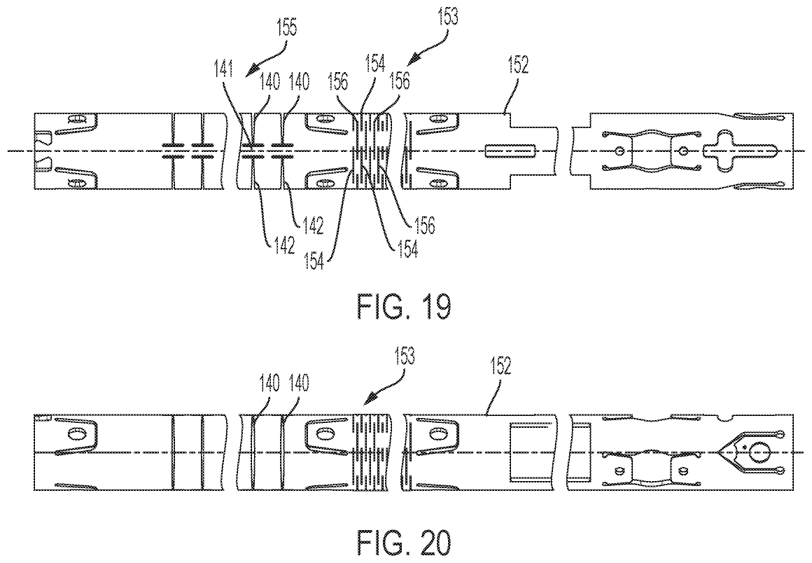

FIG. 19 illustrates an embodiment of a hypotube 152 that includes the cut pattern (marked as cut pattern 155) shown in FIGS. 15 and 16 in a distal portion of the hypotube 152, yet includes a cut pattern 153 having a greater number of directions of deflection in a proximal portion of the hypotube 152. The cut pattern 153 may be positioned proximal of the first and second sets of bi-directional cuts 140, 142. The cut pattern 153 may be positioned proximal of the cut pattern 155. The cut pattern 153 may include a plurality of longitudinally staggered cuts, forming a repeating pattern. The cuts 154, 156 of longitudinally adjacent lines of cuts may be offset by 60 degrees from each other as shown in FIG. 19, or by a different amount of offset as desired. The equally sized cuts and repeating pattern of cuts may allow for equal flexibility in all radial planes of directions. The cut pattern 153 may form an omnidirectional cut pattern. In other embodiments, flexibility may be provided in at least three directions of deflection.

The cut pattern 153 may provide for improved transmission of rotational torque from the handle 30 to the distal end of the elongate shaft 24. The cut pattern 153 may be configured to transmit torque about a longitudinal axis of the hypotube 152. Further, the cut pattern 153 may be stiffer than the cut pattern 155, at least in the two directions of flex provided by the cut pattern 155. As such, the flexibility of the elongate shaft 24 may be enhanced towards the distal end of the elongate shaft 24, which may be desirable for certain subject anatomies.

FIG. 20 illustrates a top view of the hypotube 152 shown in FIG. 19.

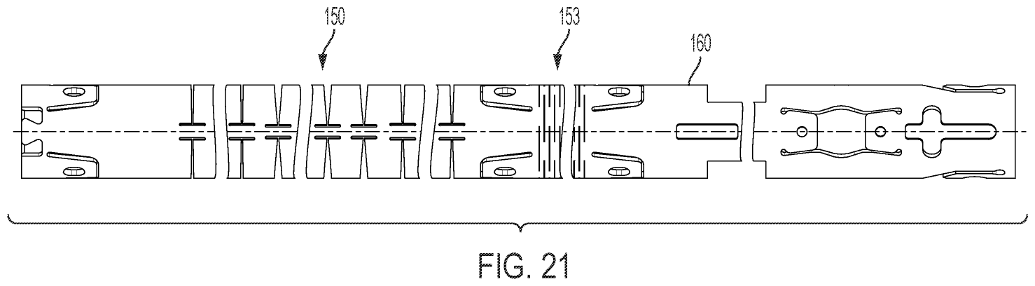

FIG. 21 illustrates a hypotube 160 including a combination of the cut pattern 153 shown in FIGS. 19 and 20 and the variably sized cut pattern 150 shown in FIG. 18.

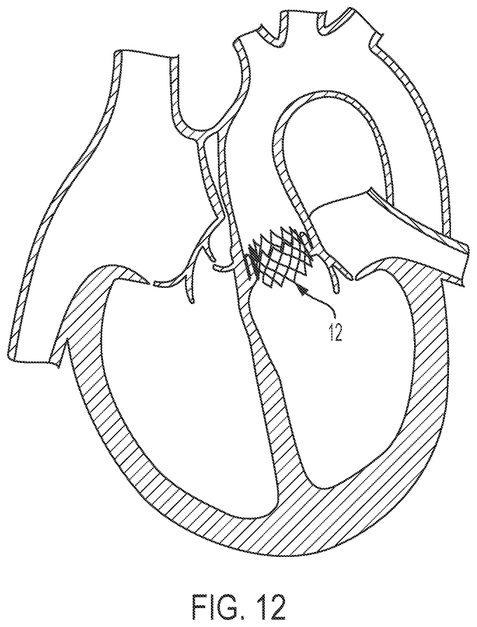

The elongate shaft 24 may be configured to be actively deflected into position utilizing any of the configurations of hypotubes disclosed herein. An implant 12 may be deployed into position as shown in FIG. 12.

A cut pattern of any or all of the hypotubes 92, 94 shown in FIG. 3 may be matched to the cut patterns of the embodiments of hypotubes shown in FIGS. 13-21. As such, a matching cut profile of the hypotubes 92, 94 may be provided. The hypotubes 92, 94 may be considered to be outer hypotubes, with the hypotubes shown in FIGS. 13-21 considered to be intermediate hypotubes.

The configuration of the hypotubes and components of the systems disclosed herein may be varied in other embodiments. Features may be combined, modified, or substituted across embodiments as desired.

The use of the hypotubes and other components disclosed herein is not limited to use with a delivery system or delivery apparatus, and may extend to use with any medical device to be inserted or withdrawn within a subject. For example, the use may extend to general medical cannula for insertion into a portion of a subject.

Hypotubes may be utilized in a variety of subjects and procedures. Subjects include (but are not limited to) medical patients, veterinary patients, animal models, cadavers, and simulators of the cardiac and vasculature system (e.g., anthropomorphic phantoms and explant tissue). Procedures include (but are not limited to) medical and training procedures.

The delivery apparatus and the systems disclosed herein may be used in transcatheter aortic valve implantation (TAVI). The delivery apparatus and the systems disclosed herein may be utilized for transarterial access, including transfemoral access, to a heart. In embodiments, various forms of implants may be delivered by a delivery apparatus utilized with system herein, such as stents or filters, or diagnostic devices, among others.

The delivery apparatuses and the systems and components disclosed herein may be utilized in transcatheter percutaneous procedures, including transarterial procedures, which may be transfemoral or transjugular. Transapical procedures, among others, may also be utilized.

Features of embodiments may be modified, substituted, excluded, or combined.

In addition, the methods herein are not limited to the methods specifically described and may include methods of utilizing the systems and apparatuses disclosed herein.

The steps of the method may be modified, excluded, or added to, with systems, apparatuses, and methods disclosed herein.

The features of the embodiments disclosed herein may be implemented independently of the delivery apparatuses, or independent of other components disclosed herein. The various apparatuses of the system may be implemented independently.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A delivery system for an implant, the delivery system comprising:
an elongate shaft having a length and having:
an implant retention area for retaining the implant,
a hypotube extending along the length of the elongate shaft and having a proximal portion and a distal portion, and including a first set of bi-directional cuts aligned longitudinally along a first side of the hypotube and a second set of bi-directional cuts aligned longitudinally along a second side of the hypotube that is opposite the first side, and two longitudinally extending spines each positioned on opposite sides of the hypotube between the first set of bi-directional cuts and the second set of bi-directional cuts, and
a pull tether extending along the length of the elongate shaft and having a distal portion and a proximal portion and an intermediate portion, with the distal portion of the pull tether coupled to a portion of the elongate shaft such that the intermediate portion of the pull tether may variably move towards the first set of bi-directional cuts or the second set of bi-directional cuts, and the pull tether configured to be pulled to deflect the hypotube.

2. The delivery system of claim 1, wherein the distal portion of the pull tether is coupled to the portion of the elongate shaft such that the intermediate portion of the pull tether is aligned with at least one of the two longitudinally extending spines.

3. The delivery system of claim 1, wherein the distal portion of the pull tether is coupled to a point on the distal portion of the hypotube that is longitudinally aligned with one of the two longitudinally extending spines.

4. The delivery system of claim 3, wherein the point is on a sidewall of the hypotube.

5. The delivery system of claim 1, wherein the hypotube is configured to deflect in two directions in a single plane.

6. The delivery system of claim 1, wherein the hypotube includes a cut pattern providing at least three directions of deflection positioned proximal of the first set of bi-directional cuts and the second set of bi-directional cuts.

7. The delivery system of claim 6, wherein the cut pattern that provides at least three directions of deflection includes a plurality of longitudinally staggered cuts.

8. The delivery system of claim 6, wherein the cut pattern that provides at least three directions of deflection is configured to provide equal flexibility of the hypotube in all radial planes of deflection.

9. The delivery system of claim 8, wherein the cut pattern that provides at least three directions of deflection is configured to transmit torque about a longitudinal axis of the hypotube.

10. The delivery system of claim 1, wherein the first set of bi-directional cuts and the second set of bi-directional cuts are each configured to have varied cut thicknesses.

11. The delivery system of claim 1, wherein the first set of bi-directional cuts and the second set of bi-directional cuts are each configured to have cuts curved such that an entire interior area of the cuts contacts simultaneously.

12. The delivery system of claim 1, wherein the implant retention area is positioned distal of the distal portion of the hypotube.

13. The delivery system of claim 1, wherein the proximal portion of the pull tether is coupled to a deflection mechanism configured to pull the pull tether to deflect the hypotube.

14. The delivery system of claim 13, further comprising a handle coupled to a proximal portion of the elongate shaft.

15. The delivery system of claim 1, further comprising a release mechanism configured to release the implant from the implant retention area.

16. A delivery system for an implant, the delivery system comprising:
an elongate shaft having a length and having:
an implant retention area for retaining the implant,
a first hypotube extending along the length of the elongate shaft and including a proximal end and a distal end, and a plurality of cuts configured to allow the first hypotube to deflect in a first direction,
a second hypotube including a distal portion and a proximal portion, the distal portion extending over the first hypotube and including a plurality of cuts configured to allow the second hypotube to deflect in a second direction that is opposite the first direction, and the proximal portion being positioned proximal of the distal end of the first hypotube and including a plurality of cuts configured to allow the proximal portion to deflect in both the first direction and the second direction.

17. The delivery system of claim 16, wherein the first hypotube is configured to impede the distal portion of the second hypotube from deflecting in the first direction.

18. The delivery system of claim 16, wherein the first hypotube is coupled to a pull tether configured to be pulled proximally to deflect the second hypotube.

19. The delivery system of claim 18, wherein the distal end of the first hypotube is coupled to a distal end of the second hypotube.

20. The delivery system of claim 16, wherein the plurality of cuts of the proximal portion of the second hypotube include a first set of bi-directional cuts aligned longitudinally along a first side of the second hypotube and a second set of bi-directional cuts aligned longitudinally along a second side of the second hypotube that is opposite the first side, and two longitudinally extending spines are each positioned on opposite sides of the second hypotube between the first set of bi-directional cuts and the second set of bi-directional cuts.

21. The delivery system of claim 20, wherein the plurality of cuts of the proximal portion of the second hypotube are positioned distal a portion of the elongate shaft that has a greater stiffness than the proximal portion of the second hypotube.

22. The delivery system of claim 21, wherein the plurality of cuts of the proximal portion of the second hypotube are positioned distal a torque shaft configured to move a sheath for covering the implant retention area.

23. The delivery system of claim 22, further comprising a release mechanism configured to rotate the torque shaft.

24. The delivery system of claim 16, wherein the plurality of cuts of the distal portion of the second hypotube include a plurality of teeth configured to fit into recesses.

25. The delivery system of claim 16, wherein the first hypotube includes a single spine positioned between the plurality of cuts of the first hypotube, and the distal portion of the second hypotube includes a single spine positioned between the plurality of cuts of the second hypotube at a position that is 180 degrees from the single spine of the first hypotube.
